(19) United States
(12) Patent Application Publication
(10) Pub. No.: US 2026/0192123 A1
(43) Pub. Date: Jul. 9, 2026

(54) A DEVICE ARRANGED FOR RADIATION HEAT TREATMENT OF PROSTATE TISSUE

(71) Applicant: PROSTALUND AB, LUND (SE)

(72) Inventors: Sonny SCHELIN, ROCKNEBY (SE); Johan WENNERHOLM, LOMMA (SE)

(21) Appl. No.: 19/128,399

(22) PCT Filed: Nov. 9, 2023
(86) PCT No.: PCT/SE2023/051133

§ 371 Date: May 8, 2025

(30) Foreign Application Priority Data
Nov. 11, 2022 (SE) 2251317-0

(51) Int. Cl. A61N 5/02

(52) U.S. Cl. CPC A61N 5/022

(57) ABSTRACT

The present invention describes a device arranged for radiation heat treatment of prostate tissue, said device utilizing a software unit being arranged for performing a method comprising the following steps:—determining the radiation energy radiated by the radiation source at given time points during a set time range;—calculating the energy point at each time point as a value of radiation energy/time, wherein 1 energy point equals a level of radiation effect/treatment time, e.g. equals 10 Watt in 1 minute treatment; calculating an accumulated energy point value for each time point;—comparing the accumulated energy point value against a given maximum energy point value for a certain prostate size;—stopping the radiation heat treatment when the accumulated energy point value is ≥ the given maximum value for a certain prostate size.

### FIELD OF THE INVENTION

The present invention relates to a device arranged for radiation heat treatment of prostate tissue, said device involving a software unit being arranged for performing a method utilized for ensuring to stop the radiation heat treatment at the right time.

### TECHNICAL BACKGROUND

The present invention is directed to a device comprising a software unit which ensures that the device stops a radiation heat treatment of prostate tissue on the right time, that is to set a so called endpoint of the radiation heat treatment.

As will be understood from the below, the device according to the present invention may utilize multiple endpoints to decide on a “real” endpoint based on different types of methodology. One aim of the present invention is to provide an optimization for setting a real endpoint for a radiation heat treatment of prostate tissue regardless of if one or multiple endpoint technologies are being utilized.

### SUMMARY OF THE INVENTION

The stated purpose above is achieved by a device arranged for radiation heat treatment of prostate tissue, said device utilizing a software unit being arranged for performing a method comprising the following steps:
determining the radiation energy radiated by the radiation source at given time points during a set time range;
calculating the energy point at each time point as a value of radiation energy/time, wherein 1 energy point equals a level of radiation effect/treatment time, e.g. equals 10 Watt in 1 minute treatment;
calculating an accumulated energy point value for each time point;
comparing the accumulated energy point value against a given maximum energy point value for a certain prostate size;
stopping the radiation heat treatment when the accumulated energy point value is ≥ the given maximum value for a certain prostate size. As should be understood, the method according to the present invention is directed to calculating an accumulated energy point value based on a certain prostate size and comparing the same to a maximum energy point value set based on that prostate size and the radiation energy/time based on a set treatment program.

Furthermore, energy points may be set as e.g. 1 energy point equals 10 Watt in 1 minute treatment.

The interaction between a user and the device may work according to the following. A certain prostate size, e.g. volume-based on a set height, length and width of the prostate, is an input provided in a user interface of the user device. The device then provides a suitable radiation treatment program (radiation energy vs time during the treatment). This is also then the input to perform the steps of i) determining the radiation energy at given time points during a set time range; and ii) calculating the energy point at each time point as a value of radiation energy/time. Based on this, the software calculates a so called energy point at each time point during the treatment, and thus calculates an accumulated energy point value. This value is compared to a set maximum energy point value and once the value is at the same level as the maximum energy point value, if it reaches the same (will be discussed further below), then the treatment is stopped, or at least paused.

If and when the accumulated energy point value reaches the set maximum, then the device ensures that the radiation treatment is stopped. The user may be able to override this to continue the radiation treatment for a short further time length, but the standard is that such an event stops the entire radiation treatment.

In relation to above it should be noted that the given maximum energy point value for a certain prostate size may be set based on a programmed curve for different prostate volume sizes, which are software data being part of the platform inside of the software unit in the device according to the present invention.

### SPECIFIC EMBODIMENTS OF THE INVENTION

Below there are provided some specific embodiments of the present invention.

According to one embodiment, the software unit also functions as a regulation unit of the device and sets the radiation energy to be provided from the device at the given time points based on the certain prostate size. As hinted above, this may be set by a programmed relationship for different prostate sizes and suitable radiation treatment programs. As an example in line with this, according to one embodiment of the present invention, the given maximum energy point value for a certain prostate size is given based on a set maximum curve based on a function for accumulated energy point value against prostate size.

Moreover, according to yet another embodiment, the software unit is arranged for also performing the steps of:
calculating cell kill of the prostate tissue and comparing a calculated cell kill value with a given maximum cell kill value; and
stopping the radiation heat treatment when the calculated cell kill value is ≥ the given maximum cell kill value.

When combining both alternatives above, then the method according to the present invention utilizes multiple endpoints based on different calculation methods and methodologies to decide when the treatment should be stopped.

According to one embodiment, the given maximum cell kill value is 20%. According to yet another embodiment, the cell kill is calculated based on Henriques' damage integral. Jung's compartment model may also be used, either alone or in combination with Henriques' damage integral.

Moreover, according to one embodiment, the radiation heat treatment is a microwave thermotherapy treatment.

Furthermore, according to yet another embodiment, stopping the radiation heat treatment is performed when any, or both, of:
the accumulated energy point value is ≥ the given maximum value for a certain prostate size; and
the calculated cell kill value is ≥ the given maximum cell kill value is fulfilled.

This embodiment implies utilizing a primary and secondary endpoint to decide on when the treatment should be stopped.

The present invention also provides a method for performing a radiation heat treatment session of a prostate of a patient, said method comprising the following steps:
providing a prostate size value of the patient into a device according to the present invention,
performing a radiation heat treatment of the prostate of the patient until the device stops the radiation heat treatment where the software unit of the device performs the steps according to the present invention; and
then evaluating the situation and either ending the radiation heat treatment session or initiating an extra radiation heat treatment session.

To use the accumulated energy point value in accordance with the present invention brings about advantages during the radiation heat treatment on a prostate. By using only the mentioned cell kill value above presupposes a perfect placement of a used IP sensor and also perfect anesthesia with adrenaline for the treatment. By using also the accumulated energy point value according to the present invention, these perfect placement and anesthesia with adrenaline are not as crucial. Therefore, a device according to the present invention provides a secure way of ensuring an optimal radiation heat treatment for a prostate where the risk for a too short or too long treatment is minimized.

### DETAILED DESCRIPTION OF THE DRAWINGS

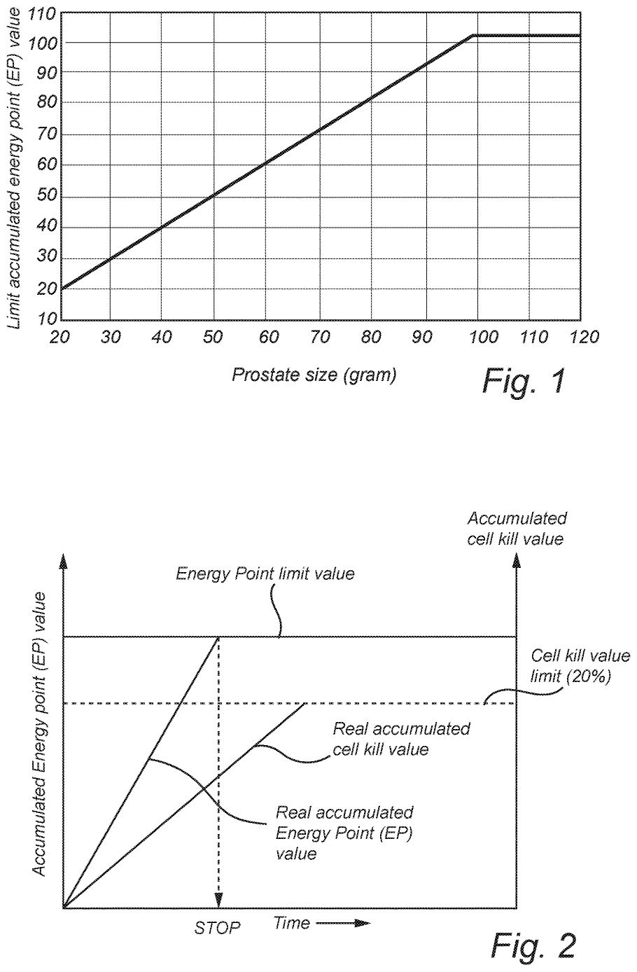

In FIG. 1 there is shown a graph visualizing one possible relationship between a suitable limit value for an accumulated energy point (EP) value and a prostate size for a prostate intended to be treated. This graph may act as one relationship being part of the software of a device according to the present invention.

Moreover, in FIG. 2 there is shown a visualization of a general function of a software of a device according to one embodiment of the present invention. In this case and this embodiment there is shown that the device provides two set limit values, namely one for the energy point value and one for the cell kill value, the latter being normally 20%. Then the software operates by calculating the real values of both these values during the radiation heat treatment of a prostate tissue. When one of the limit values is reached, then this triggers the device to stop the treatment.

1. A device arranged for radiation heat treatment of prostate tissue, said device utilizing a software unit being arranged for performing a method comprising the following steps:
determining the radiation energy radiated by the radiation source at given time points during a set time range;
calculating the energy point at each time point as a value of radiation energy/time, wherein 1 energy point equals a level of radiation effect/treatment time;
calculating an accumulated energy point value for each time point;
comparing the accumulated energy point value against a given maximum energy point value for a certain prostate size;
stopping the radiation heat treatment when the accumulated energy point value is ≥ the given maximum value for a certain prostate size.

2. The device according to claim 1, wherein the software unit also functions as a regulation unit of the device and sets the radiation energy to be provided from the device at the given time points based on the certain prostate size.

3. The device according to claim 1, wherein the given maximum energy point value for a certain prostate size is given based on a set maximum curve based on a function for accumulated energy point value against prostate size.

4. The device according claim 1, wherein the software unit is arranged for also performing the steps of:
calculating cell kill of the prostate tissue and comparing a calculated cell kill value with a given maximum cell kill value; and
stopping the radiation heat treatment when the calculated cell kill value is ≥ the given maximum cell kill value.

5. The device according to claim 4, wherein the given maximum cell kill value is 20%.

6. The device according to claim 4, wherein the cell kill is calculated based on Henriques' damage integral.

7. The device according to claim 1, wherein the radiation heat treatment is a microwave thermotherapy treatment.

8. The device according to claim 3, wherein stopping the radiation heat treatment is performed when any, or both, of:
the accumulated energy point value is ≥ the given maximum value for a certain prostate size; and
the calculated cell kill value is ≥ the given maximum cell kill value is fulfilled.

9. A method for performing a radiation heat treatment session of a prostate of a patient, said method comprising the following steps:
providing a prostate size value of the patient into a device according to claim 1,
performing a radiation heat treatment of the prostate of the patient until the device stops the radiation heat treatment where the software unit of the device performs the steps according to claim 1; and
then evaluating the situation and either ending the radiation heat treatment session or initiating an extra radiation heat treatment session.
